(19) United States
(12) Patent Application Publication
(10) Pub. No.: US 2020/0260675 A1
(43) Pub. Date: Aug. 20, 2020

(54) SYSTEM FOR DRY ARTIFICIAL POLLINATION OF CULTIVATED TREES OR SHRUBS BY INSECT-BORNE POLLEN AND METHOD OF DOING THE SAME

(71) Applicant: EDETE PRECISION TECHNOLOGIES FOR AGRICULTURE LTD., Qiryat Tivon (IL)

(72) Inventors: Eylam RAN, Qiryat Tivon (IL); Asaf Menachem BORENSTEIN, Haifa (IL)

(21) Appl. No.: 16/644,900

(22) PCT Filed: Sep. 6, 2018
(86) PCT No.: PCT/IL2018/051017

§ 371 Date: Mar. 5, 2020

Related U.S. Application Data
(60) Provisional application No. 62/555,057, filed on Sep. 7, 2017.
(60) Provisional application No. 62/582,350, filed on Nov. 7, 2017.
(60) Provisional application No. 62/584,928, filed on Nov. 13, 2017.

(51) Int. Cl. A01H 1/02

(52) U.S. Cl. CPC A01H 1/025

(57) ABSTRACT

A system for dry artificial pollination of cultivated trees or shrubs by insect-borne pollen comprises: (a) an air supply for generating an air flow; (b) a container accommodating pollen grains and maintaining the pollen grains in fluidized condition; (c) a high voltage power supply; (d) at least two electrostatic pollinators for directing the pollen grains carried air flow in a direction of cultivated trees or shrubs; the at least two electrostatic pollinators being in fluid connection with the container; (e) a feeder interconnecting the container and the at least electrostatic pollinator; the feeder configured for feeding the pollen grains in a fluidized condition from the container into the at least one electrostatic pollinator; the feeder comprises a doser configured for dispensing a predetermined amounts of the pollen grains in fluidized condition; (f) a distributer configured for segmenting and distributing the pollen grains in a fluidized condition to at least two the electrostatic pollinators. The system comprises a mixer configured for atomizing the pollen grains.

### FIELD OF THE INVENTION

The present invention relates to artificial pollination and, more particularly, to devices and methods implementing artificial pollination by dry insect-borne pollen.

### BACKGROUND OF THE INVENTION

Pollination is the transfer of pollen from the anther, the male parts of the flower, to the female part, where fertilization occurs, resulting in the reproduction of seeds, fruits and vegetables. Pollination is done either by wind or by animals, mainly insects. Nature's preference for genetic diversification requires cross pollination-a delivery of pollen from one flower to a flower on another plant of the same species. Cross pollination is key for quality and quantity of crops. In agriculture pollination management, various verities of the same crop are interplanted in order to get synchronized blooms allowing the transfer of pollen for cross pollination. About 75% of the world's crops rely on animal pollination.

The main agriculture pollinators, by far, are domesticated honeybees. The honeybee, Apis mellifera, has been the dominant pollinator for decades but is now threatened by pesticides, pathogens, parasites and poor nutrition. Beekeepers around the world suffer loses of 15-40 percent of their managed honeybee colonies annually due to the above-mentioned reasons. Other wild insects are declining in number and diversity. Honeybees require optimal environmental conditions in order to pollinate, that prevent bees from getting agricultural optimal yield.

Constant increase of the world's population combined with higher income levels, results in a growing demand for food. Agriculture is driven by slow but constant arable land growth from 1.4 Billion Hectares on 1961 to more than 1.6 billion hectares by 2016, vast intensive monocultures resulting in increase of yield. The described continues growth is heavily dependent on honeybees for pollination and due to the above-mentioned weakening of the honeybees' population is resulting in increase of the costs of pollination.

The mechanized pollination system described hereafter can solve the dependence of agricultural yield on honeybees and other insects, ensure food security by insuring and growing the yield by providing an efficient optimal pollination. Moreover, the mechanized pollination system will solve cross pollination problems resulting by desynchronized bloom of different varieties and guaranty the agricultural yield.

There are attempts to mechanized pollination of insect-borne pollen by creating a mixing the pollen with a liquid. Creating pollen slurry that is sprayed in the form of droplets, micro-droplets or mist onto the trees.

However, there is evidence that pollination by dispersing f pollen slurry can causes rot, increase fungus accumulation and damage to the tree and or flowers. Further, there is evidence that pollination by dispensing of pollen slurry is of limited efficacy.

There is a long-felt need of providing a mechanized system and a method of pollination of insect-borne pollen that does not require admixing the pollen to a liquid. In other words, there is a need. a system dispensing dry insect-borne pollen.

Mechanized pollination of wind-borne pollen is known in the art. It is typically achieved with blowers or applicators.

In general, wind-borne pollen is adapted in various ways to maximize dispersal in air. Wind-borne pollen is also expected to disperse as dry, non-adhesive isolated grains, and to have a smooth rather than slippery surface.

However, insect-borne pollen typically has adhesive qualities, conferred by a lipid coating (Pollenkitt) on the pollen grains. Insect-borne pollen are typically non-aerodynamic, sticky and tend to conglomerate. Consequently, the grains disperse as heavier ‘clumps’ rather than individually.

There is a long-felt and unmet need for a system and method that can handle and disperse insect-borne-pollen in an air flow, like wind-borne-pollen, a system that will overcome the natural properties of the insect-borne-pollen of size, non-aerodynamic shape, stickiness and tendency to conglomerate.

### SUMMARY OF THE INVENTION

It is hence one object of the invention to disclose a system for dry artificial pollination of cultivated trees or shrubs by insect-borne pollen comprising: (a) an air supply for generating an air flow; (b) a container accommodating pollen grains and maintaining said pollen grains in fluidized condition; (c) a high voltage power supply; (d) at least two electrostatic pollinators for directing said pollen grains carried air flow in a direction of cultivated trees or shrubs; said at least two electrostatic pollinators being in fluid connection with said container; (e) a feeder interconnecting said container and said at least electrostatic pollinator; said feeder configured for feeding said pollen grains in a fluidized condition from said container into said at least one electrostatic pollinator; said feeder comprises a doser configured for dispensing a predetermined amounts of said pollen grains in fluidized condition; (f) a distributer configured for segmenting and distributing the said pollen grains in a fluidized condition to at least two said electrostatic pollinators.

It is a core purpose of the invention to provide the system comprising a mixer configured for atomizing said pollen grains.

Another object of the invention is to disclose at least one electrostatic pollinator comprising a conduit for guiding said air flow mixed with said pollen mixture in a direction of said cultivated trees or shrubs.

A further object of the invention is to disclose at least one electrostatic pollinator comprising a corona-discharge electrode for charging said pollen mixture in said direction of said cultivated trees or shrubs; said corona-discharge electrode is electrically connected to high voltage power supply.

A further object of the invention is to disclose at least one electrostatic pollinator based on tribo-charging.

A further object of the invention is to disclose the electrified electrode which is an electrically conductive grid connected to said high voltage power supply.

A further object of the invention is to disclose the system comprising a feeding system including: a feeder, a mixer and tubes connecting the container to the feeder, the feeder to the mixer and the mixer to the distributer, wherein air from said air supply is fed into at least one of the said feeding system components.

A further object of the invention is to disclose the system comprising a transport arrangement configured for supporting said at least one electrostatic pollinator near said cultivated trees or shrubs at a predetermined distance.

A further object of the invention is to disclose the system comprising a chassis carrying said transport arrangement.

A further object of the invention is to disclose the system comprising said chassis carrying said transport arrangement is self-propelled and self-steering.

A further object of the invention is to disclose the transport arrangement having a telescopic structure.

A further object of the invention is to disclose the transport arrangement having an articulated structure.

A further object of the invention is to disclose a method of artificially pollinating cultivated by insect-borne pollen. The aforesaid method comprises steps of: (a) providing dry insect borne pollen of cultivated trees or shrubs; (b) providing a system for artificial pollination; said system comprising at least two electrostatic pollinators further comprising: (i) an air supply for generating an air flow; (ii) a container accommodating pollen grains and maintaining said pollen grains in fluidized condition; (iii) a high voltage power supply; (iv) at least one electrostatic pollinator for directing said pollen grains carried air flow in a direction of cultivated trees or shrubs; said at least one electrostatic pollinator being in fluid connection with said container; (v) a feeder interconnecting said container and said at least electrostatic pollinator; said feeder configured for feeding said pollen grains in a fluidized condition from said container into said at least one electrostatic pollinator; said feeder comprises a doser configured for dispensing a predetermined amounts of said pollen grains in fluidized condition and a mixer configured for atomizing said pollen grains; (vi) a distributer configured for segmenting and distributing the said pollen grains in a fluidized condition to at least two said electrostatic pollinators; (b) pointing said at least one electrostatic pollinator to said cultivated plant; (c) generating said air flow by said air supply; (d) providing said air flow to said feeder and mixer; (e) guiding said pollen grains in said fluidized condition in a direction of cultivated trees or shrubs; (f) charging said dry insect-borne pollen grains in said fluidized condition in said direction of said cultivated plant.

A further object of the invention is to disclose a system for dry artificial pollination of cultivated trees or shrubs by insect-borne pollen comprising: (a) an air supply for generating an air flow; (b) a container accommodating pollen grains and maintaining said pollen grains in fluidized condition; (b) a high voltage power supply; (c) at least one electrostatic pollinator for charging said pollen grains carried air flow in a direction of cultivated trees or shrubs; said at least one electrostatic pollinator being in fluid connection with said container; (d) a feeder interconnecting said container and said at least electrostatic pollinator; said feeder configured for feeding said pollen grains in a fluidized condition from said container into said at least one electrostatic pollinator; said feeder comprises a doser configured for dispensing a predetermined amounts of said pollen grains in fluidized condition and a mixer configured for atomizing said pollen grains; (e) a distributer configured for segmenting and distributing the said pollen grains in a fluidized condition to at least two said electrostatic pollinators; (f) at least one sensing unit configured for detecting at least one spatial parameter; (g) at least one sensing unit configured for sensing neighboring environment; (h) a control unit configured for receiving spatial parameters and parameters of neighboring environment from said sensing units.

It is a core purpose of the invention to provide the control unit configured for recognizing geometry of said cultivated trees or shrubs and pointing said at least one electrostatic pollinator thereto such that a flow of said pollen grains in fluidized condition created by said at least one electrostatic pollinator compensates wind magnitude within said geometry and create an volume of substantially still air therewithin.

A further object of the invention is to disclose at least one sensing unit comprising a module configured for recognizing said cultivated trees or shrubs.

A further object of the invention is to disclose at least one sensing unit comprising a meteorological module configured for sensing at least one meteorological parameter of environmental air.

A further object of the invention is to disclose at least one meteorological parameter selected from the group consisting of wind velocity, wind direction, temperature and relative humidity and any combination thereof.

A further object of the invention is to disclose at least one sensing unit comprising a spatial sensor configured for determining a geographic position of said system.

A further object of the invention is to disclose the control unit configured for calculating geometry of said cultivated plant on the basis of measurements obtained by said at least one spatial sensor.

A further object of the invention is to disclose the control unit configured for calculating flower coverage of said cultivated trees or shrubs on the basis of images obtained by said at least one sensing unit.

A further object of the invention is to disclose the control unit configured for regularly interrogating said at least one sensing unit.

A further object of the invention is to disclose the control unit configured for time closed loop control in real time.

A further object of the invention is to disclose the control unit configured for controlling at least one parameter selected from the group consisting of a flow velocity of said pollen grains within said at least one electrostatic pollinator, a voltage on an electrode within said electrostatic pollinator, a dispensable dose of said pollen grains and any combination thereof.

A further object of the invention is to disclose the control unit configured for controlling at least one parameter selected from the group consisting of a distance between said electrostatic pollinator and said cultivated trees or shrubs, a direction of a flow of said pollen grains, a position of said system relative to said cultivated trees or shrubs and any combination thereof.

A further object of the invention is to disclose the system comprising two self-propelled and self-steering portions; said portions comprise said at least one electrostatic pollinator each.

A further object of the invention is to disclose the pollinators cooperatively positionable such that said volume of substantially still air is created.

A further object of the invention is to disclose a method of dry artificial pollination of cultivated trees or shrubs by insect-borne pollen. The foresaid method comprises steps of: (a) providing dry insect borne pollen of cultivated trees or shrubs; (b) providing a system for artificial pollination; said system comprising at least one electrostatic pollinator further comprising: (i) an air supply for generating an air flow; (ii) a container accommodating pollen grains and maintaining said pollen grains in fluidized condition; (iii) a high voltage power supply; (iv) at least one electrostatic pollinator for charging said pollen grains carried air flow in a direction of cultivated trees or shrubs; said at least one electrostatic pollinator being in fluid connection with said container; (v) a feeder interconnecting said container and said at least electrostatic pollinator; said feeder configured for feeding said pollen grains in a fluidized condition from said container into said at least one electrostatic pollinator; said feeder comprises a doser configured for dispensing a predetermined amounts of said pollen grains in fluidized condition and a mixer configured for atomizing said pollen grains; (vi) a distributer configured for segmenting and distributing the said pollen grains in a fluidized condition to at least two said electrostatic pollinators; (vii) at least one sensing unit configured for detecting spatial parameters of said system and sensing neighboring environment; (viii) a control unit configured for receiving spatial parameters and parameters of neighboring environment from said sensing unit; said control unit is configured for recognizing a geometry of said cultivated trees or shrubs and pointing said at least one electrostatic pollinator thereto such that pollen grains in fluidized condition reach cultivated trees or shrubs; (c) for detecting a geographic position said system and sensing neighboring environment; (d) receiving geographic position and parameters of neighboring environment from said sensing unit; (e) recognizing a geometry of said cultivated trees or shrubs; (f) pointing said at least one electrostatic pollinator to said cultivated plant; (g) generating said air flow by said air supply; (h) providing said air flow to said feeding system; (i) fluidizing said pollen grains within said container; (j) guiding said pollen grains in said fluidized condition in a direction of cultivated trees or shrubs; (k) charging said pollen grains in said fluidized condition in said direction of said cultivated plant by said electrified electrode.

### BRIEF DESCRIPTION OF THE DRAWINGS

In order to understand the invention and to see how it may be implemented in practice, a plurality of embodiments is adapted to now be described, by way of non-limiting example only, with reference to the accompanying drawings, in which

FIG. 1 is an external view of a system for dry artificial pollination of cultivated trees or shrubs by insect-borne pollen;

FIG. 2 is a functional block diagram of a system for dry artificial pollination of cultivated trees or shrubs by insect-borne pollen;

FIG. 3 is a schematic view of an electrostatic pollinator;

FIGS. 4a and 4b are schematic side and top views presenting an area to be pollinated within a cultivated plant;

FIGS. 5a and 5b are schematic presentations illustrating calculation of tree geometry;

FIGS. 6a and 6b are schematic views illustrating different flower coverage of a cultivated plant;

FIG. 7 is a schematic presentation of exemplary trajectory of a system for dry artificial pollination of cultivated trees or shrubs by insect-borne pollen during operation;

FIGS. 8a and 8b are side views of alternative embodiments the present invention;

FIGS. 9a and 9b illustrate untrimmed and trimmed cultivated trees or shrubs to be pollinated;

FIG. 10 is a schematic view of an air-permeable shade net over cultivated trees or shrubs;

FIG. 11 is a block diagram which illustrates functioning a control unit;

FIG. 12 is a flowchart of navigation of a system for dry artificial pollination of cultivated trees or shrubs by insect-borne pollen during operation;

FIG. 13 is a flowchart of maneuvering a system for dry artificial pollination of cultivated trees or shrubs by insect-borne pollen during operation;

FIGS. 14a and 14b are schematic views indicating symmetrical and asymmetrical areas of steady wind compensation within a cultivated plant by two electrostatic pollinators;

FIGS. 15 is a schematic view illustrating cycling a flow of pollen grains within a cultivated plant;

FIGS. 16a and 16b are schematic views indicating symmetrical and asymmetrical arrangements of steady wind compensation within a cultivated plant by two electrostatic pollinators.

### DETAILED DESCRIPTION OF THE INVENTION

The following description is provided, so as to enable any person skilled in the art to make use of said invention and sets forth the best modes contemplated by the inventor of carrying out this invention. Various modifications, however, are adapted to remain apparent to those skilled in the art, since the generic principles of the present invention have been defined specifically to provide a system for dry artificial pollination of cultivated trees or shrubs by insect-borne pollen and a method of implementing the same.

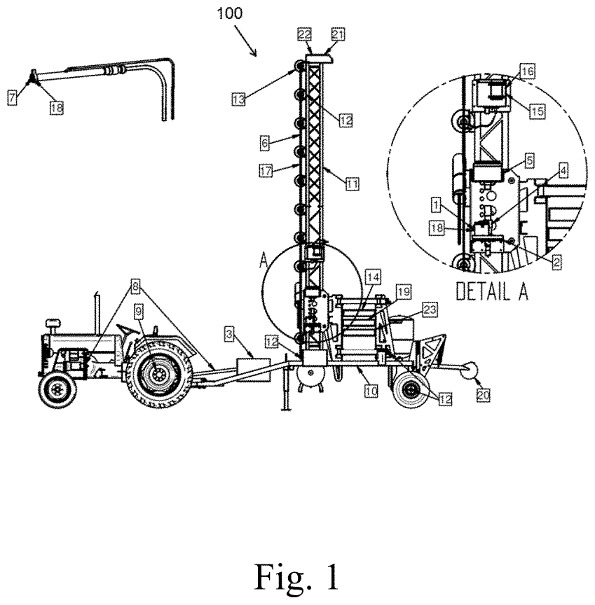

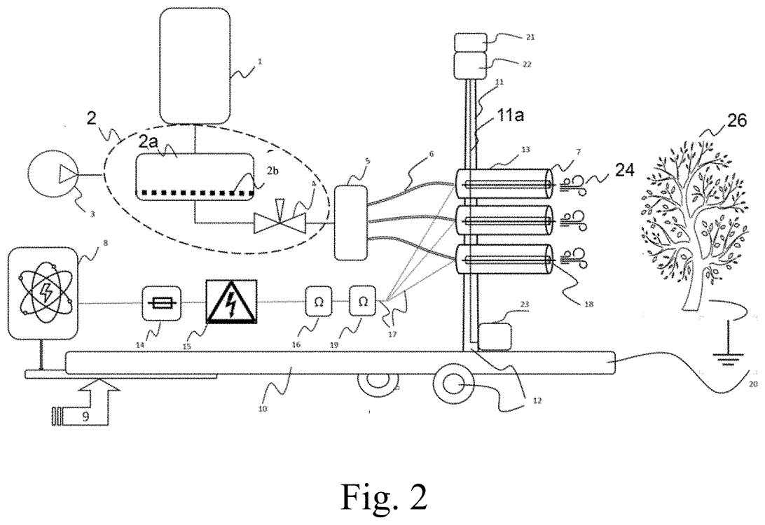

Reference is now made FIGS. 1 and 2 presenting an external view and a functional diagram of system 100 for dry artificial pollination of cultivated trees or shrubs by insect-borne pollen, respectively. Air supply 3 feeds compressed air to feeding system 2 accommodating insect-borne pollen grains. Then, the pollen grains gravitationally move to feeding system 2 (detailed description will be provided below). Feeding system 2 includes feeder 2a and doser 2b and mixer 4. Feeder 2a is provided with a stirrer (not shown) assisting for uniformly filling an internal space of the feeder, preventing the pollen grains from aggregation and their adhesion to internal walls of the feeder 2a. The pollen grains are moved by compressed air or by venturi effect via the mixer 4 such that pollen grains are mixed with the compressed air in a homogenous manner. Then air-pollen grain mixture is fed to distributer 5 which is configured for distributing the aforesaid air-pollen grain mixture over nozzle 7 via pipes 6. Numerals 13 and 18 refer to external shields and central electrodes, respectively. Electrically charged pollen cloud 24 is directed to a geometrical area 26 recognized by system 100 to be cultivated. Electrical charging of the pollen grains can be performed by at least one alternative such as charging in container 1, by corona discharge by electrode 18 and by a tribo effect based on friction. System 100 is mounted on chassis 10 which can be self-propelled or manually movable. In the case of the self-propelled embodiment, the system is provided with a propulsion system (not shown). Numeral 8 refers to an autonomous power supply. Electric circuitry is energized via circuit breaker 14, converter 15, high voltage distribution unit 16 and high voltage safety unit 19, and conduction system 17. A plurality of electrostatic pollinators organized into an array is also in the scope of the present invention. Transport arrangement 11 is configured for mounting an array of electrostatic pollinators and sensing units of meteorological variables and spatial parameters 21 and 22, respectively. Numeral 11a refers to a data bus between sensing units 21 and 22 and data processing unit 23. Unit 20 of potential equalization should be adapted for different types of ground. Unit 21 is configured for sensing meteorological variables such as wind velocity and direction, air temperature, relative humidity and luminance. Unit 22 is configured for identifying pollination targets and relative position of a pollination target to a pollinator and building a 3D model of a pollination target. Processing unit 23 is configured for said control unit is configured for controlling at least one parameter selected from the group consisting of a flow velocity of said pollen grains within said at least one electrostatic pollinator, a voltage on an electrode within said electrostatic pollinator, a dispensable dose of said pollen grains, a distance between said electrostatic pollinator and said cultivated trees or shrubs, a direction of a flow of said pollen grains, a position of said system relative to said cultivated trees or shrubs.

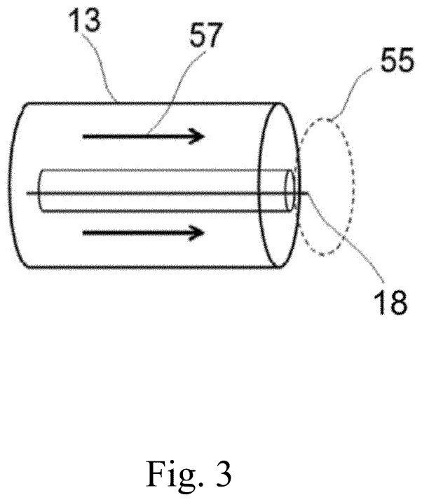

Reference is now made to FIG. 3 schematically presenting an electrostatic pollinator. Arrows 57 indicate a flow of pollen grains within tubal shield 13. Electrode 18 which is electrified by high voltage charges the flow of pollen grains which forms an electrically charged cloud 55 of pollen grains in proximity of a pollination target.

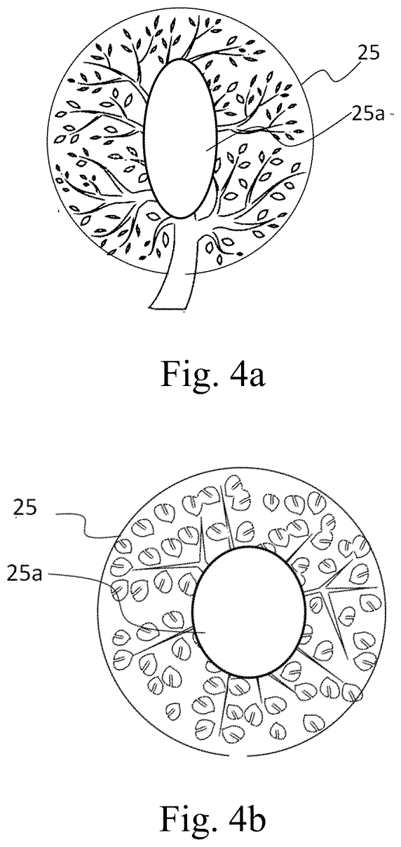

Reference is now made to FIGS. 4a and 4b presenting schematic cross-sectional side and top views, respectively, of geometry of an area to be pollinated. As described above, the control unit is configured for building a 3D geometric model of the area to be pollinated. Tree geometry 25 is defined based on data from spatial sensors. Location of volume of substantially still air 25a is calculated by controller.

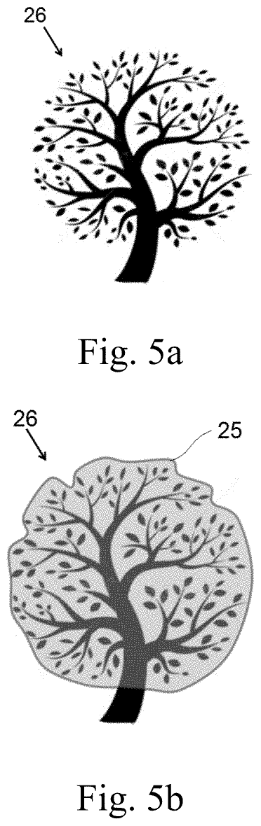

Reference is now made to FIGS. 5a and 5b presenting schematic cross-sectional side views of a cultivated plant 26 and a tree geometry 25 which geometrically defines the volume to be pollinated.

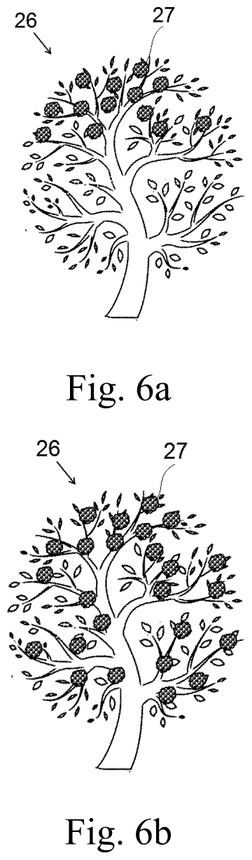

Reference is now made to FIGS. 6a and 6b, presenting schematic side views of a cultivated plant. FIG. 6a relates to a cultivated plant characterized by substantially uniform flower coverage, FIG. 6b is covered by flowers 27 on its top only.

In an embodiment, the optimal pollination is achieved at an optimal distance range from the pollinator and minimal wind velocity in proximity to a pollination target. The optimal distance range and wind velocity are provided by sensing a position of the pollination target and meteorological variables and optimally positioning the pollinator relative the pollination target at an optimal distance range such that a flow of pollen grains dispensed by the pollinator compensates wind velocity and creates a volume of still air.

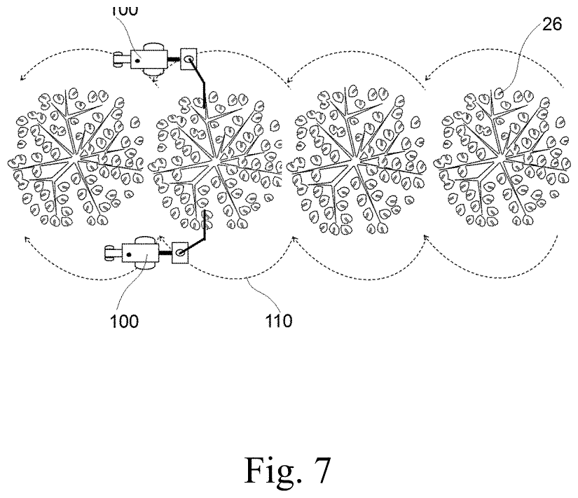

Reference is now made to FIG. 7 illustrating field use pollinating system 100. The aforesaid systems are shown in an orchard. Arches 110 show trajectory of maneuvering pollinating systems 100. This trajectory is directed to keeping the distance between pollinating systems 100 and an area to be pollinated of cultivated trees or shrubs 26 optimal.

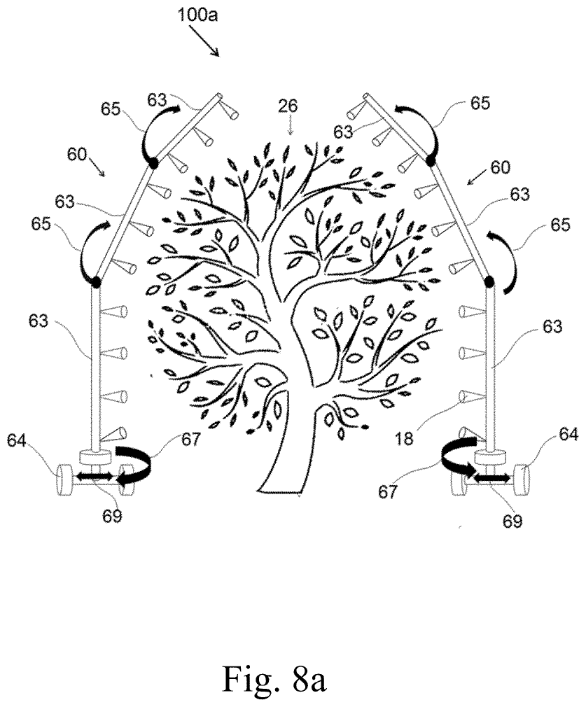

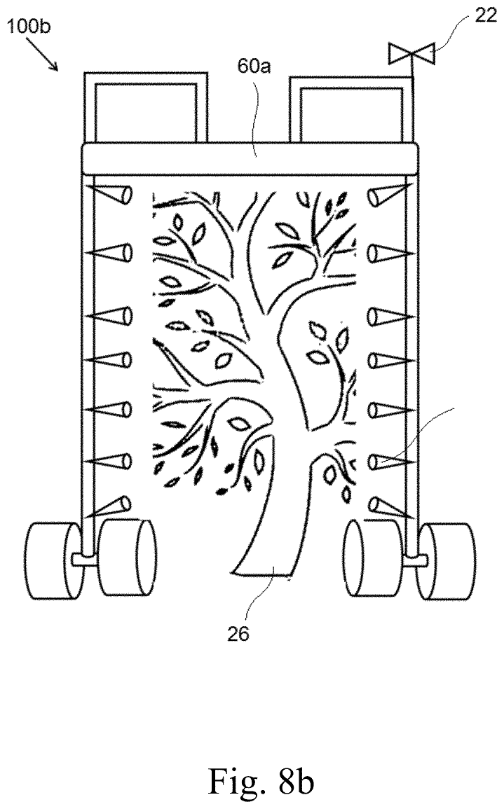

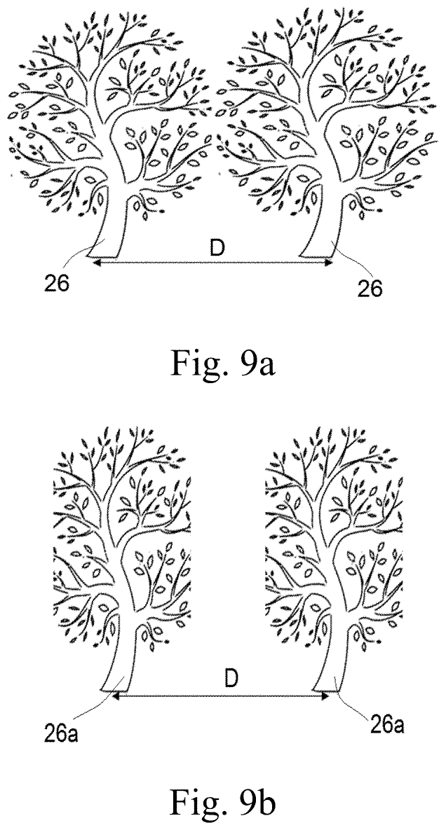

Reference is now made to FIGS. 8 and 9 presenting alternative embodiments 100a and 100b of the present invention. System 100a is provided with an articulated transport arrangement 60 comprising members 63 hingedly interconnected to each other. Arrows 65 indicate a direction of manipulating members 63 in order to provide the minimal distance to the pollination area. In other words, the transport arrangements “embrace” cultivated plant 26. Arrow 69 indicate additional freedom degrees which can be used for sake of minimization of the distance to the pollination area. Arrow 67 indicate the swivel capability of the transport arrangement 60 to allow the system to create the counter-wind vector for the volume of substantially still air.

Numeral 64 refers to a transport wheel.

Embodiment 100b has doubled transport arrangement 60a receiving a cultivated plant 26 thereinto.

Reference is now made to FIGS. 9a and 9b presenting two rows of cultivated trees spaced apart from each other at distance D. In FIG. 9a, untrimmed cultivated trees 26 are shown while, in FIG. 9b, trimmed cultivated trees are presented.

Embodiment 100a (FIG. 8a) is designed for untrimmed cultivated trees (FIG. 9a), Embodiment 100b (FIG. 8b) is designed for the trimmed cultivated trees (FIG. 9b)

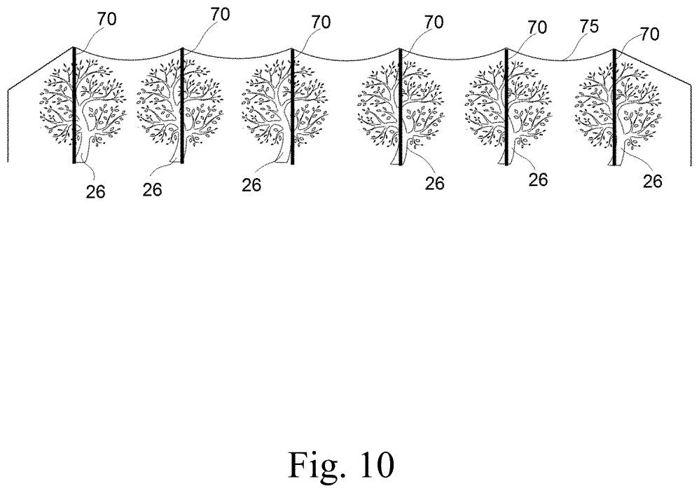

Reference is now made to FIG. 10 presenting a protective net 75 permeable to air and sunlight. the protective net 75 supported by pillars 70 covers a row of cultivated trees 26. The height of the protective net 75 is adapted for operation of a system for dry artificial pollination of cultivated trees or shrubs by insect-borne pollen under the net.

Protective nets are optional only in a non-insects environment and therefore this invention is unique by allowing it also for insect-pollinated cultivars.

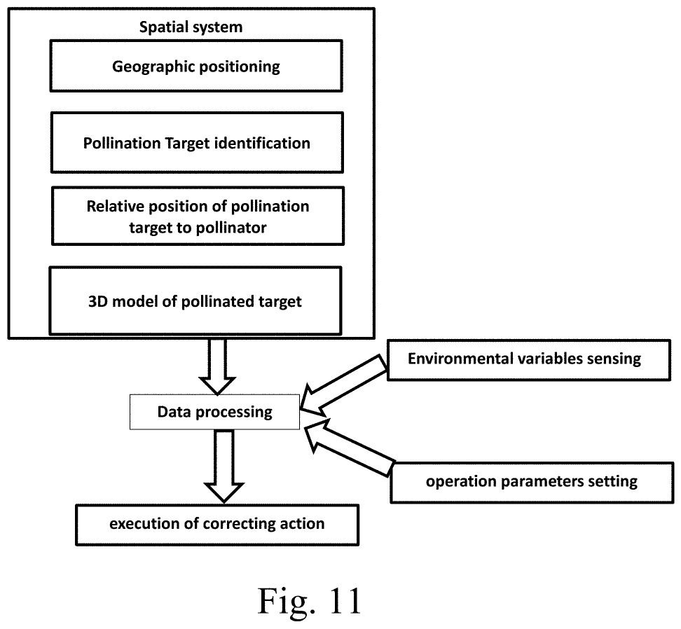

Reference is now made to FIG. 11 presenting a schematic block-diagram of sensing-and-controlling part of the system for dry artificial pollination of cultivated trees or shrubs by insect-borne pollen. The sensing part comprises environmental sensing unit 21 and spatial sensing unit 22. The aforesaid unit 22 is configured for geographic positioning (22a), identifying a pollination target (22b), determining a relative position of a pollinator relative to a pollination target (22c) and building a 3D model of a pollination target (22d). All obtained data received from units 21 and 22 are analyzed in control unit 23. Operation parameters 21a of the system are modified by an actuator 23a which is controlled by control unit 23. In an embodiment, the spatial system includes additional sensors.

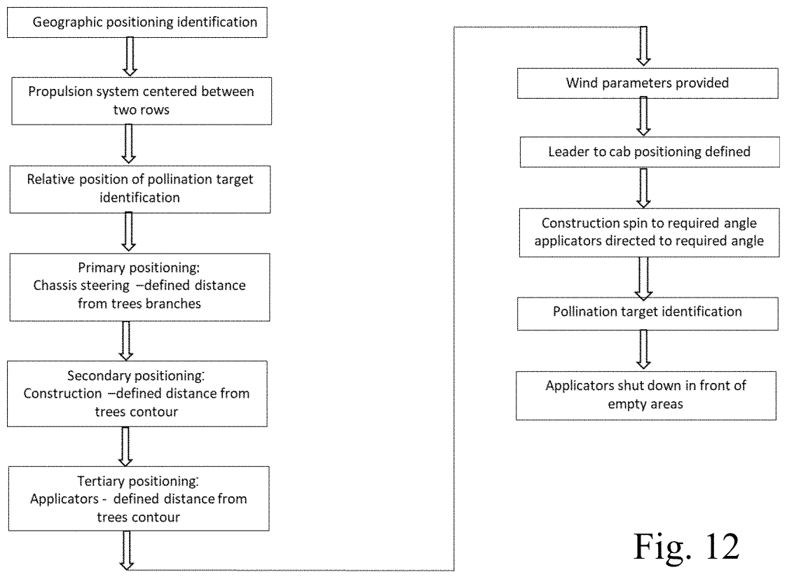

Reference is now made to FIG. 12 presenting a flowchart of operation of the sensing-and-control part of the system. In the beginning of this procedure, a geographic position of the system is identified by means of a GPS sensor. At step 210, the system maneuvers in order to position the system is an optimal location between two rows of cultivated trees or shrubs. Relative position of a pollination target is determined at step 220. Then, the chassis maneuvers in order to take an optimal position (step 230). The transport arrangement carrying at least one electrostatic pollinator also is also optimally positioned relative to identified geometry of an area to be pollinated (step 240). Individual pollinators are manipulated at step 250. Steps 210 to 240 are performed on the basis of data obtained by spatial sensing unit. At step 260, environmental variables such as wind velocity are provided by environmental sensing unit. If the system includes a leader portion and a cab portion, their mutual position is determined at step 270 by means of a leader transmitter. The transport arrangement is manipulated in order to place it into the position defined by the control unit (step 280). An image analysis unit identifies the pollination target (step 290). After pollination of the identified area, the electrostatic pollinators are shut down by the control unit (step 300).

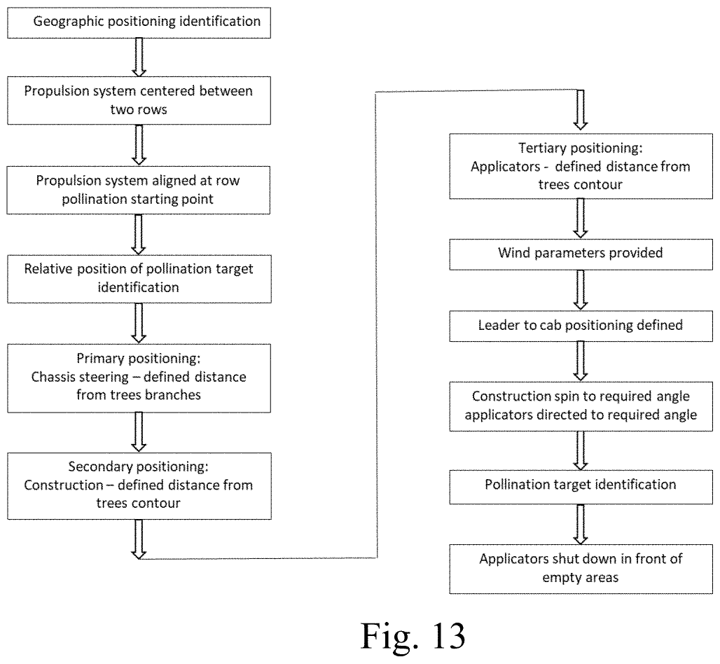

Reference is now made to FIG. 13 presenting a flowchart of maneuvering the leader-cab system. Addressing to FIG. 12, steps 200, 210 and 230 to 300 are disclosed previously. At additional step 320, the system is positioned at a row pollination start point. Steps 200, 270, 280 and 320 are performed by the leader positioning unit. In this case, the cab position is determined relative to the leader. Steps 210 to 250, 290 and 300 are performed the leader and cab separately. Step 260 is performed by the leader only.

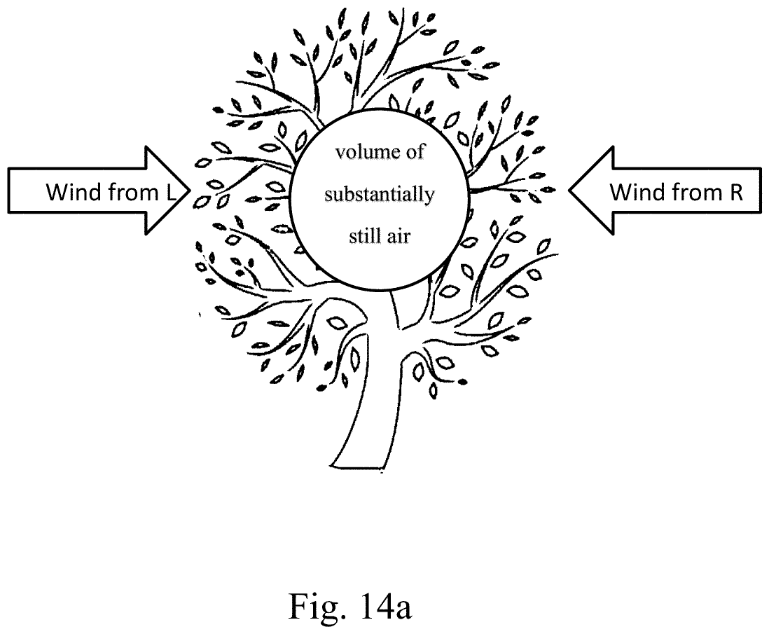

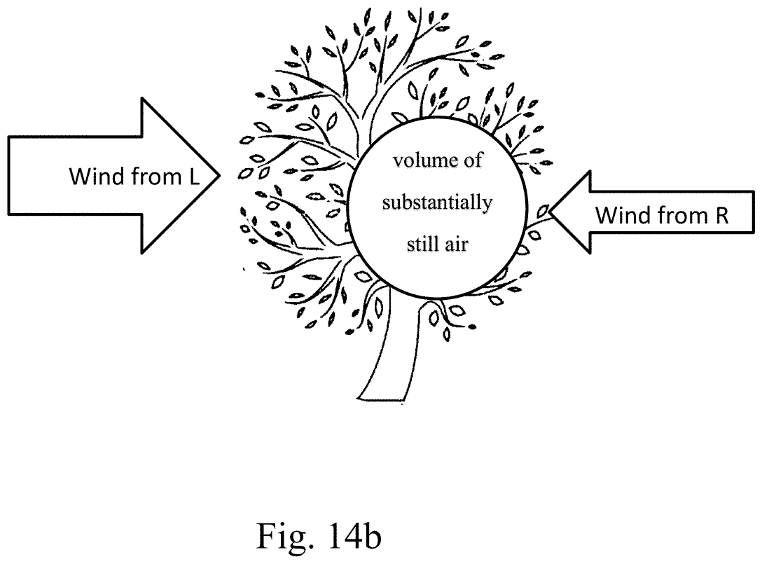

Reference is now made to FIGS. 14a and 14b illustrating a procedure of creating an area of still air. FIG. 14a shows creating a symmetric area of still air. Airflows from the left and the right are approximately equal. In FIG. 14b, the airflows from the left and the right are not equal and the area of still air has an asymmetric position relative to cultivated plant.

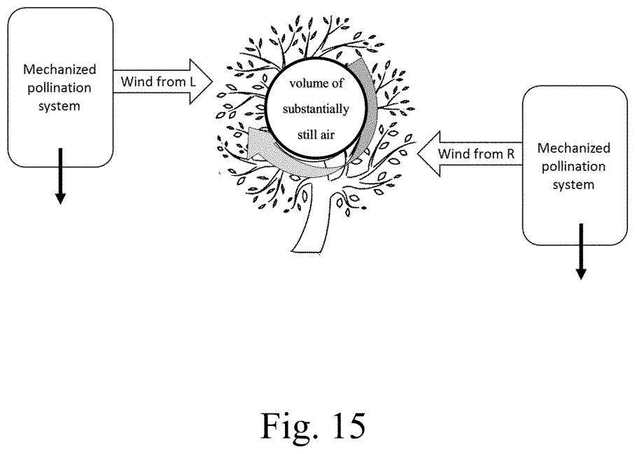

Reference is now made to FIG. 15 presenting an arrangement of pollination system where the pollen grains are dispensed from two pollination systems located in an opposite manner relative to the cultivated plant. The pollen grains are dispensed at different heights. As a result, there is air flow circulation within a crown of the cultivated plant.

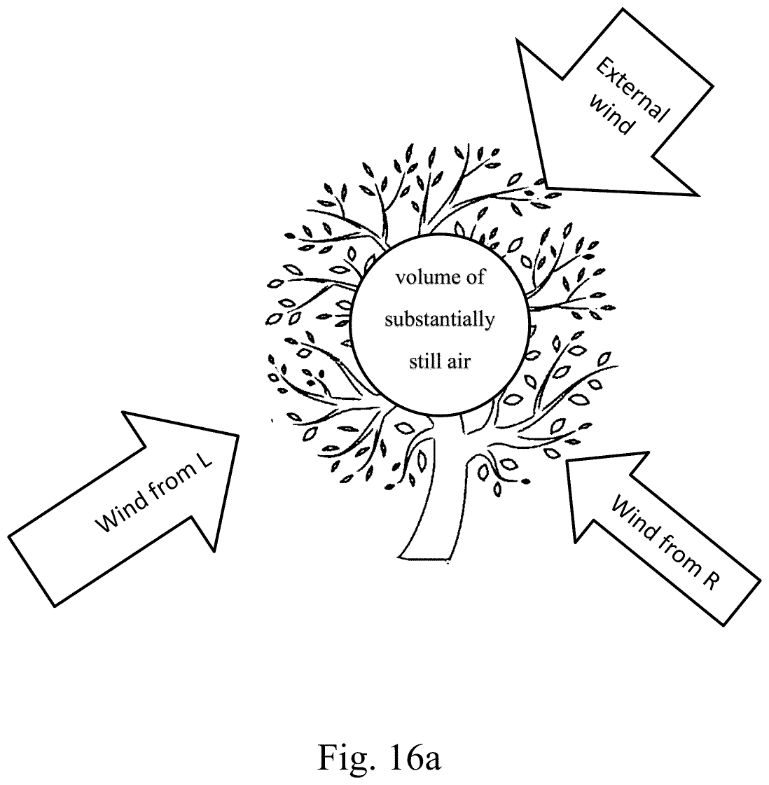

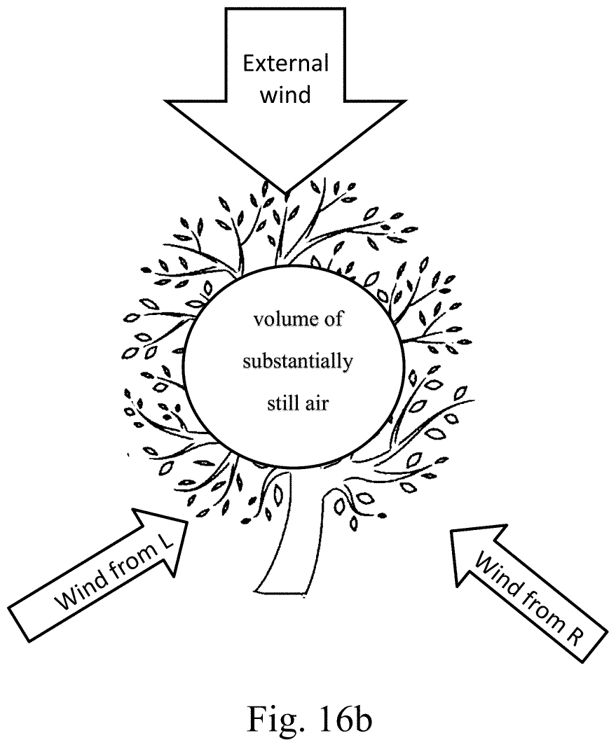

Reference sis now made to FIGS. 16a and 16b presenting alternative arrangements for creating an area of still air. In FIG. 16a, an external wind is compensated by two air flows symmetrically arranged relative to the external wind. FIG. 16b shows an asymmetrical arrangement.

1.-28. (canceled)

29. A system for dry artificial pollination of insect-pollinated trees or shrubs comprising:
a. a container configured to accommodate dry pollen grains for at least one insect-pollinated tree or shrub and to maintain said pollen grains in fluidized condition;
b. at least two electrostatic pollinators, each of said at least two electrostatic pollinators configured to induce an electrostatic charge on said pollen grains; and
c. a feeder interconnecting said container and said at least two electrostatic pollinators; said feeder comprising a doser;
wherein said system further comprises at least one air supply configured to generate at least two convergent air flows, each of said at least two air flows in fluid communication with said doser, said doser configured to distribute said pollen grains in charged fluidized condition into at least one of said at least two air flows; each said air flow comprising said pollen grains in said charged fluidized condition; each said air flow configured to direct said pollen grains in said charged fluidized condition in a direction of said at least one insect-pollinated tree or shrub;
wherein, by convergence of said at least two air flows, a volume of substantially still air comprising said pollen grains in said charged fluidized condition is generable;
further wherein said pollination is dry pollination using said pollen grains.

30. The system according to claim 29, wherein at least one of said at least two electrostatic pollinators further comprises a conduit for guiding said air flow mixed with said pollen grains in a direction of said at least one insect-pollinated tree or shrub.

31. The system according to claim 29, wherein at least one of said at least two electrostatic pollinators comprises at least one electrode in electrical connection with at least one high voltage power supply, said least one electrode being a corona-discharge electrode for charging said pollen grains in said direction of said at least one insect-pollinated tree or shrub.

32. The system according to claim 29, wherein said at least one of said at least two electrostatic pollinators is based on tribo-charging.

33. The system according to claim 29, additionally comprising a feeding system including: a feeder, a mixer and tubes connecting the container to the feeder, the feeder to the mixer and the mixer to the distributer, wherein air from said air supply is fed into at least one of the components of said feeding system.

34. The system according to claim 29, additionally comprising a transport arrangement configured to support at least one of said at least two electrostatic pollinators near said at least one insect-pollinated tree or shrub at a predetermined distance.

35. A method of dry artificial pollination of insect-pollinated trees or shrubs by insect-borne pollen; said method comprising steps of:
a. providing dry pollen grains for at least one insect-pollinated tree or shrub;
b. providing a system for artificial pollination; said system comprising:
i. a container configured to accommodate said pollen grains and to maintain said pollen grains in fluidized condition;
ii. at least two electrostatic pollinators; each of said at least two electrostatic pollinators being in fluid connection with a container; each of said at least two electrostatic pollinators is configured to induce an electrostatic charge on said pollen grains;
iii. a feeder interconnecting said container and said at least two electrostatic pollinators; said feeder comprising a closer; and
iv. at least one air supply configured to generate at least two convergent air flows, each of said at least two air flows in fluid communication with at least one said doser, said doser configured to distribute said pollen grains in charged fluidized condition into at least one of said at least two air flows; each of said at least two air flows comprising said pollen grains in said charged fluidized condition; each said at least two air flows configured to direct said pollen grains in said charged fluidized condition in a direction of said at least one insect-pollinated tree or shrub;
c. pointing said at least two air flows convergently at said at least one insect-pollinated tree or shrub;
d. said at least two electrostatic pollinators electrostatically charging said pollen grains;
e. said doser distributing said pollen grains in said charged fluidized condition into at least one of said at least two air flows; each of said at least two air flows comprising said pollen grains in said charged fluidized condition; and
f. said at least two air flows directing said pollen grains in said charged fluidized condition in a direction of said at least one insect-pollinated tree or shrub;
thereby, by said convergence of said air flows, generating a volume of substantially still air comprising said pollen grains in said charged fluidized condition at said at least one insect-pollinated tree or shrub; and
thereby pollinating said at least one insect-pollinated tree or shrub with said pollen grains.

36. A system for dry artificial pollination of insect-pollinated trees or shrubs comprising:
a. an air supply for generating an air flow;
b. a container configured to accommodate dry pollen grains for at least one insect-pollinated tree or shrub and to maintain said pollen grains in fluidized condition;
c. a high voltage power supply;
d. at least one electrostatic pollinator for charging said pollen grains carried in said air flow in a direction of at least one insect-pollinated tree or shrub; said at least one electrostatic pollinator being in fluid connection with said container;
e. a feeder interconnecting said container and said at least one electrostatic pollinator; said feeder configured to feed said pollen grains in a fluidized condition from said container into said at least one electrostatic pollinator; said feeder comprises a doser configured to dispense a predetermined amount of said pollen grains in fluidized condition and a mixer configured to atomize said pollen grains;
f. a distributer configured to segment and to distribute said pollen grains in a fluidized condition to said at least one electrostatic pollinator;
g. at least one sensing unit configured to detect at least one spatial parameter;
h. at least one sensing unit configured to sense a neighboring environment; and
i. a control unit configured to receive said at least one spatial parameter and parameters of said neighboring environment from said at least one sensing unit;
wherein said control unit is configured to recognize geometry of said at least one insect-pollinated tree or shrub and pointing said at least one electrostatic pollinator thereto such that a flow of said pollen grains in fluidized condition created by said at least one electrostatic pollinator compensates wind magnitude within said geometry and create a volume of substantially still air therewithin.

37. The system according to claim 36, wherein said at least one electrostatic pollinator comprises a corona-discharge electrode for charging said pollen grains; said corona-discharge electrode is electrically connected to high voltage power supply.

38. The system according to claim 36, wherein said at least one electrostatic pollinator is based on tribo-charging.

39. The system according to claim 36 wherein said at least one sensing unit comprises a module configured to recognize said at least one insect-pollinated tree or shrub.

40. The system according to claim 39, wherein said at least one sensing unit comprises a meteorological module configured to sense at least one meteorological parameter of environmental air.

41. The system according to claim 36, wherein said at least one meteorological parameter is selected from the group consisting of wind velocity, wind direction, temperature and relative humidity and any combination thereof.

42. The system according to claim 36, wherein said at least one sensing unit comprises a spatial sensor configured to determine a geographic position of said system.

43. The system according to claim 36, wherein said control unit is configured to calculate geometry of said cultivated plant on the basis of measurements obtained by said at least one spatial sensor.

44. The system according to claim 36, wherein said control unit is configured to calculate flower coverage of said at least one insect-pollinated tree or shrub on the basis of images obtained by said at least one sensing unit.

45. The system according to claim 36, wherein said control unit is configured for time closed loop control in real time.

46. The system according to claim 36, wherein said control unit is configured to control at least one parameter selected from the group consisting of a flow velocity of said pollen grains within said at least one electrostatic pollinator, a voltage on an electrode within said electrostatic pollinator, a dispensable dose of said pollen grains and any combination thereof.

47. The system according to claim 36, wherein said control unit is configured to control at least one parameter selected from the group consisting of a distance between said electrostatic pollinator and said at least one insect-pollinated tree or shrub, a direction of a flow of said pollen grains, a position of said system relative to said at least one insect-pollinated tree or shrub and any combination thereof.

48. A method of dry artificial pollination of insect-pollinated trees or shrubs by insect-borne pollen; said method comprising steps of:
a. providing dry pollen grains for at least one insect-pollinated tree or shrub;
b. providing a system for artificial pollination; said system comprising at least one electrostatic pollinator further comprising:
i. an air supply for generating an air flow;
ii. a container configured to accommodate said pollen grains and to maintain said pollen grains in fluidized condition;
iii. a high voltage power supply;
iv. at least one electrostatic pollinator for charging said pollen grains carried air flow in a direction of said at least one insect-pollinated tree or shrub; said at least one electrostatic pollinator being in fluid connection with said container;
v. a feeder interconnecting said container and said at least electrostatic pollinator; said feeder configured to feed said pollen grains in a fluidized condition from said container into said at least one electrostatic pollinator; said feeder comprises a doser configured to dispense a predetermined amount of said pollen grains in fluidized condition and a mixer configured to atomize said pollen grains;
vi. a distributer configured to segment and to distribute said pollen grains in a fluidized condition to at least two said electrostatic pollinators;
vii. at least one sensing unit configured to detect at least one spatial parameter of said system and to sense parameters of a neighboring environment;
vii. a control unit configured to receive said at least one spatial parameter and said parameters of a neighboring environment from said sensing unit;
wherein said control unit is configured to recognize a geometry of said at least one insect-pollinated tree or shrub and to point said at least one electrostatic pollinator thereto such that said pollen grains in fluidized condition reach said at least one insect-pollinated tree or shrub;
c. for detecting a geographic position said system and sensing neighboring environment;
d. receiving geographic position and parameters of neighboring environment from said sensing unit;
e. recognizing a geometry of said at least one insect-pollinated tree or shrub;
f. pointing said at least one electrostatic pollinator at said at least one insect-pollinated tree or shrub;
g. generating said air flow by said air supply;
h. providing said air flow to said feeding system
i. fluidizing said pollen grains within said container;
j. guiding said pollen grains in said fluidized condition in a direction of said at least one insect-pollinated tree or shrub; and
k. charging said pollen grains in said fluidized condition in said direction of said at least one insect-pollinated tree or shrub by said electrified electrode.
